(19) United States
(12) Patent Application Publication
(10) Pub. No.: US 2026/0028619 A1
(43) Pub. Date: Jan. 29, 2026

(54) METHODS, COMPOSITIONS, AND KITS FOR SINGLE CELL ANALYSIS BY TAGMENTATION

(71) Applicant: 10x Genomics, Inc., Pleasanton, CA (US)

(72) Inventors: Paulius Mielinis, Stockholm (SE); Marlon Stoeckius, Stockholm (SE); David Sukovich, Oakland, CA (US); Marco Serra, Dublin, CA (US); Lauren Gutgesell, Fremont, CA (US)

(21) Appl. No.: 19/279,412

(22) Filed: Jul. 24, 2025

Related U.S. Application Data
(60) Provisional application No. 63/675,299, filed on Jul. 25, 2024.

(51) Int. Cl. C12N 15/10

(52) U.S. Cl. CPC C12N 15/1065

(57) ABSTRACT

Provided herein are methods, systems, and kits for detecting analytes of interest from biological samples using tagmentation. The methods include tagmenting DNA/RNA hybrids that include an mRNA molecule and a barcode nucleic acid molecule to generate a plurality of tagmented fragments, and separating a first tagmented fragment including an end portion of the mRNA and a barcode from other tagmented fragments.

### RELATED APPLICATION DATA

This application claims benefit of U.S. Provisional Application No. 63/675,299 filed Jul. 25, 2024, of which is herein incorporated by reference in its entirety.

### BACKGROUND

Nucleic acid sequencing is a versatile tool that helps scientists advance the understanding of biology and has wide-ranging applications in various fields, such as medical diagnostics, biotechnology, forensic biology, and virology. Reverse transcription-based assays for resolving sequences at the single cell level, typically obtain sequence information from an end (e.g., a 3′ end) of the transcript. However, typical single cell assays involve processing of full transcripts even when only a fraction (such as a 3′ end) is to be sequenced. Improved workflows with more streamlined assaying steps are therefore desirable and may be helpful in expanding the utility of single cell transcriptomic methodologies and applications thereof.

### SUMMARY

The present disclosure features improved methods, compositions, and kits for single cell sequencing of target analytes of a biological sample. The present methods, compositions, and kits for single cell transcriptomics analyte preparation can bypass full cDNA amplification, fragmentation and/or other processes required in existing workflows, thereby streamlining library construction and reducing reagent usage. More specifically, the methods include using a transposase to insert a sequencing primer sequence into a single cell-barcoded nucleic acid analyte, or an intermediate or complement thereof, to thereby generate single cell-barcoded nucleic acid fragments that are suitable for library preparation and sequence analysis.

Thus, provided herein are methods for processing an mRNA molecule, including:
(a) in a partition, hybridizing the mRNA molecule to a barcode nucleic acid molecule attached to a solid support, wherein the barcode nucleic acid molecule comprises: (i) a barcode sequence and (ii) a domain that hybridizes to a 3′ sequence of the mRNA molecule;
(b) extending the barcode nucleic acid molecule, thereby generating a barcoded nucleic acid molecule annealed to the mRNA molecule, wherein the barcoded nucleic acid molecule comprises a sequence complementary to the mRNA molecule;
(c) tagmenting the barcoded nucleic acid molecule annealed to the mRNA molecule, thereby generating a plurality of tagmented fragments comprising a first tagmented fragment, wherein the first tagmented fragment comprises a first strand comprising an mRNA fragment comprising the 3′ sequence of the mRNA molecule covalently attached to the transposon end sequence, and a second strand comprising the barcode nucleic acid molecule and a portion of the sequence complementary to the mRNA molecule; and
(d) separating the first tagmented fragment from other tagmented fragments of the plurality of tagmented fragments. In some embodiments, steps (c) and (d) are performed outside of the partition. In some embodiments, steps (b), (c), and (d) are performed outside the partition.

In some embodiments, the methods further include, after the tagmenting in (c), extending the second strand using the first strand as a template, thereby generating an extended barcoded fragment comprising: (i) the barcode sequence and (ii) a copy of 3′ mRNA fragment, and (iii) the transposon end sequence. In some embodiments, the extending the second strand is after the separating in (d).

In some embodiments, the solid support is magnetic, and the separating comprises magnetically separating. In some embodiments, the solid support comprises iron oxide.

In some embodiments, the barcode nucleic acid molecule comprises an affinity tag, and the separating comprises affinity purification. In some embodiments, the solid support comprises an affinity tag, and the separating comprises affinity purification. In some embodiments, the solid support comprises a polymer of monomers and the nucleic acid barcode molecule is covalently attached to a monomer comprising the affinity tag.

In some embodiments, the solid support is a bead. In some embodiments, the solid support comprises polyacrylamide.

In some embodiments, the method further includes: (i) hybridizing a primer to the extended barcoded fragment, and (ii) extending the primer, thereby generating a copy of the extended barcoded fragment. In some embodiments, the method further includes separating the copy of the extended barcoded fragment from the extended barcode fragment. In some embodiments, the primer comprises a sequencing primer sequence. In some embodiments, the primer comprises a sequence complementary to the transposon end sequence.

In some embodiments, the method further includes determining a sequence of the extended barcoded fragment.

In some embodiments, the method further includes sequencing the extended barcoded fragment. In some embodiments, the method further includes amplifying a copy of the extended barcoded fragment prior to the sequencing.

In some embodiments, following the (a) hybridizing, a terminal end of 3′ sequence of the mRNA molecule is not hybridized to the barcode nucleic acid molecule and after the tagmenting in (c), the method further comprises removing the terminal end of 3′ sequence that is not hybridized. In some embodiments, the terminal end is a terminal portion of a poly-A tail. In some embodiments, the terminal portion of the poly-A tail consists of between 1 and 200 A nucleotides. In some embodiments, the removing comprises treating with a nuclease. In some embodiments, the nuclease is exonuclease T or RNase T.

In some embodiments, the method further includes, after the removing, extending the tagmented mRNA fragment using the barcoded nucleic acid molecule as a template, thereby generating a barcoded extension product comprising: (i) the barcode sequence or a complement thereof, and (ii) a copy of the tagmented mRNA fragment. In some embodiments, the barcoded extension product is a RNA/DNA hybrid molecule. In some embodiments, the method further includes determining a sequence of the barcoded extension product or an intermediate thereof.

In some embodiments, the method further includes sequencing the barcoded extension product or an intermediate thereof. In some embodiments, the method further includes reverse transcribing the barcoded extension product or the intermediate thereof prior to the sequencing. In some embodiments, the method further includes amplifying the barcoded extension product or the intermediate thereof prior to the sequencing.

In some embodiments, the sequencing includes determining: (i) the barcode sequence or a complement thereof, and (ii) all or a portion of a sequence of the barcoded nucleic acid fragment or a complement thereof.

In some embodiments, the extending in (b) is using a reverse transcriptase.

In some embodiments, the tagmenting in (c) comprises contacting the barcoded nucleic acid molecule with a transposome comprising the transposon end sequence. In some embodiments, the transposome comprises a transposase. In some embodiments, the transposase is a Tn5 transposase, a Mu transposase, a Tn7 transposase, a Vibrio species transposase, or a functional derivative thereof.

In some embodiments, the barcoded extension product comprises the transposon end sequence at a 3′ end of the barcoded extension product.

In some embodiments, the transposon end sequence comprises a sequencing primer sequence. In some embodiments, the sequencing primer sequence is a P7 sequence.

In some embodiments, the tagmenting in (c) is performed in a presence of a permeabilization agent. In some embodiments, the permeabilization agent comprises a detergent.

In some embodiments, the method further comprises prior to (a), partitioning a cell, cell bead, or nucleus comprising the mRNA analyte with the nucleic acid barcode molecule attached to the solid support. In some embodiments, the partition comprises a well. In some embodiments, the partition comprises a droplet.

In some embodiments, the barcoded nucleic acid molecule is a partially double-stranded RNA/DNA molecule. In some embodiments, the partially double-stranded RNA/DNA molecule comprises an RNA sequence of the nucleic acid analyte and a DNA sequence complementary to the sequence of the nucleic acid analyte.

In some embodiments, barcode sequence comprises a partition-specific barcode sequence.

In some embodiments, the nucleic acid barcode molecule further comprises one or more functional domains, a unique molecular identifier, or a combination thereof. In some embodiments, the one or more functional domains comprises a sequencing primer sequence.

In some aspects, the present disclosure provides a system comprising: (a) an mRNA analyte (b) a nucleic acid barcode molecule comprising: (i) a barcode sequence and (ii) a domain that hybridizes to a 3′ sequence of the mRNA analyte, wherein the nucleic acid barcode molecule is attached to a solid support; and (c) a transposome comprising a transposase and a transposon end sequence, wherein the transposon end sequence comprises a sequencing primer sequence. In some embodiments, the solid support comprises a bead. In some embodiments, the solid support comprises hydrogel. In some embodiments, the transposase is a Tn5 transposase, a Mu transposase, a Tn7 transposase, a Vibrio species transposase, or a functional derivative thereof. In some embodiments, the transposome comprises two transposon end sequences. In some embodiments, the system further comprises a reverse transcriptase. In some embodiments, the system further comprises a polymerase. In some embodiments, the polymerase is a DNA polymerase. In some embodiments, the system further comprises a plurality of dNTPs. In some embodiments, the system further comprises one or more permeabilization reagents. In some embodiments, the one or more permeabilization reagents comprises a protease, a surfactant, or a detergent. In some embodiments, the protease comprises Proteinase K, pepsin, or collagenase. In some embodiments, the system further comprises an RNase. In some embodiments, the system further comprises a DNase.

In some aspects, provided herein is a kit comprising: (a) nucleic acid barcode molecule comprising: (i) a barcode sequence and (ii) a domain that hybridizes to an mRNA analyte, wherein the nucleic acid barcode molecule is attached to a solid support; (b) a reverse transcriptase; (c) a transposome comprising a transposase and a transposon end sequence, wherein the transposon end sequence comprises a sequencing primer sequence; and (d) instructions for performing a method as provided herein. In some embodiments, the solid support comprises a hydrogel. In some embodiments, the system further comprises solid support comprises a bead. In some embodiments, the transposase is a Tn5 transposase, a Mu transposase, a Tn7 transposase, a Vibrio species transposase, or a functional derivative thereof. In some embodiments, the transposome comprises two transposon end sequences. In some embodiments, the kit comprises the reverse transcriptase. In some embodiments, the kit comprises the polymerase. In some embodiments, the polymerase is a DNA polymerase. In some embodiments, the kit further comprises a plurality of dNTPs. In some embodiments, the kit further comprises one or more permeabilization reagents. In some embodiments, the one or more permeabilization reagents comprises a protease, a surfactant, or a detergent. In some embodiments, the kit further comprises an RNase. In some embodiments, the kit further comprises a DNase. In some embodiments, the domain that hybridizes to an mRNA analyte comprises a polyT sequence.

### BRIEF DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an example of a microfluidic channel structure for partitioning individual analyte carriers.

FIG. 2 shows an exemplary microfluidic channel structure for delivering barcode-carrying beads to droplets.

FIG. 3 shows an example of a microfluidic channel structure for the controlled partitioning of beads into discrete droplets.

FIG. 4 illustrates an example of a barcode-carrying bead.

FIG. 5 illustrates another example of a barcode-carrying bead.

FIG. 6 schematically illustrates an example microwell array.

FIG. 7 schematically illustrates an example workflow for processing nucleic acid molecules.

FIG. 8 schematically shows another example of a barcode-carrying bead.

FIG. 9 shows an example of tagmentation-based single cell assay method as provided herein.

FIG. 10 shows an additional example of a tagmentation-based single cell assay method as provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise. “A and/or B” is used herein to include all of the following alternatives: “A,” “B”, “A or B”, and “A and B”.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value or rounded to the nearest significant figure, in all cases inclusive of the provided value.

Headings, e.g., (a), (b), (i) etc., are presented merely for case of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

The term “barcode,” as used herein, generally refers to a label, or identifier, which conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include polynucleotide barcodes, random nucleic acid and/or amino acid sequences, and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses). The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.; as well as invertebrates, such as annelids, echinoderms, cnidarians, gastropods, crustaceans, cephalopods, mollusks, Porifera sponges, arachnids, and insects.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. The nucleic acid barcode molecule may be coupled to or attached to the nucleic acid molecule comprising the nucleic acid sequence. For example, a nucleic acid barcode molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, which can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a check swab. The sample may be a plasma or serum sample. The sample may be a cell-free or cell-free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle” may be used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a nucleus of a cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell but may not include other constituents of the cell. An example of such constituents is a nucleus or an organelle. A cell may be a live cell. The live cell may be capable of being cultured, for example, being cultured when enclosed in a gel or polymer matrix or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that is less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), RNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition can be a physical container, compartment, or vessel, such as a droplet, a flowcell, a reaction chamber, a reaction compartment, a tube, a well, or a microwell. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

### I. Overview

The present disclosure features improved methods, compositions, and kits for the single cell detection of target analytes from a biological sample. The present methods, compositions, and kits for preparing mRNA analytes for single cell 3′ sequencing, can streamline library construction and reducing reagent usage. More specifically, the present methods include using a transposase to insert a sequencing primer sequence into single cell barcoded mRNA analyte, or an intermediate or complement thereof while the mRNA analyte is associated with a solid support, and separating the solid support and associated nucleic acid molecules from unbound nucleic acid molecules prior to further extensions, amplifications, and other library preparation steps.

### II. Single Cell Tagmentation-Based Workflows

In some aspects, provided herein are single cell tagmentation-based approaches for generating an RNA transcript sequencing library (and methods for sequencing). These approaches include methods involving tagmentation of a double-stranded RNA/cDNA hybrid molecule including an RNA analyte end (e.g., a 3′ end) in proximity to a single cell barcode, tagmenting the double-stranded RNA/cDNA hybrid, and separating a fragment including the RNA analyte end and the single cell barcode from other fragments prior to downstream processing steps such as second strand synthesis and amplification.

In some embodiments, provided herein is a method for processing an mRNA molecule, including:
(a) in a partition, hybridizing the mRNA molecule to a barcode nucleic acid molecule attached to a solid support, wherein the barcode nucleic acid molecule comprises: (i) a barcode sequence and (ii) a domain that hybridizes to a 3′ sequence of the mRNA molecule;
(b) extending the barcode nucleic acid molecule, thereby generating a barcoded nucleic acid molecule annealed to the mRNA molecule, wherein the barcoded nucleic acid molecule comprises a sequence complementary to the mRNA molecule;
(c) tagmenting the barcoded nucleic acid molecule annealed to the mRNA molecule, thereby generating a plurality of tagmented fragments comprising a first tagmented fragment, wherein the first tagmented fragment comprises a first strand comprising 3′ sequence of the mRNA molecule covalently attached to the transposon end sequence, and a second strand comprising the barcode nucleic acid molecule and a portion of the sequence complementary to the mRNA molecule; and
(d) separating the first fragment from other fragments of the plurality of tagmented fragments.

In some embodiments, steps (c) and (d) are performed outside of the partition. In some embodiments, steps (b), (c), and (d) are performed outside the partition.

In some embodiments, the methods further include, after the tagmenting in (c), extending the second strand using the first strand as a template, thereby generating an extended barcoded fragment comprising: (i) the barcode sequence and (ii) a copy of 3′ mRNA fragment, and (iii) the transposon end sequence. In some embodiments, the extending the second strand is after the separating in (d).

In some embodiments, the method further includes: (i) hybridizing a primer to the extended barcoded fragment, and (ii) extending the primer, thereby generating a copy of the extended barcoded fragment. In some embodiments, the method further includes separating the copy of the extended barcoded fragment from the extended barcode fragment. In some embodiments, the primer comprises a sequencing primer sequence. In some embodiments, the primer comprises a sequence complementary to the transposon end sequence.

In some embodiments, the method further includes determining a sequence of the extended barcoded fragment.

In some embodiments, the method further includes sequencing the extended barcoded fragment. In some embodiments, the method further includes amplifying a copy of the extended barcoded fragment prior to the sequencing.

In some embodiments, following the (a) hybridizing, a terminal end of 3′ sequence of the mRNA molecule is not hybridized to the barcode nucleic acid molecule and after the tagmenting in (c), the method further comprises removing the terminal end of 3′ sequence that is not hybridized. In some embodiments, the terminal end is a terminal portion of a poly-A tail. In some embodiments, the terminal portion of the poly-A tail consists of between 1 and 200 A nucleotides. In some embodiments, the removing comprises treating with a nuclease. In some embodiments, the nuclease is exonuclease T or RNase T.

In some embodiments, the method further includes, after the removing, extending the tagmented mRNA fragment using the barcoded nucleic acid molecule as a template, thereby generating a barcoded extension product comprising: (i) the barcode sequence or a complement thereof, and (ii) a copy of the tagmented mRNA fragment. In some embodiments, the barcoded extension product is an RNA/DNA hybrid molecule. In some embodiments, the method further includes determining a sequence of the barcoded extension product or an intermediate thereof.

In some embodiments, the method further includes sequencing the barcoded extension product or an intermediate thereof. In some embodiments, the method further includes reverse transcribing the barcoded extension product or the intermediate thereof prior to the sequencing. In some embodiments, the method further includes amplifying the barcoded extension product or the intermediate thereof prior to the sequencing.

In some embodiments, the sequencing includes determining: (i) the barcode sequence or a complement thereof, and (ii) all or a portion of a sequence of the barcoded nucleic acid fragment or a complement thereof.

In some embodiments, the extending in (b) is using a reverse transcriptase.

In some embodiments, the tagmenting in (c) comprises contacting the barcoded nucleic acid molecule with a transposome comprising the transposon end sequence. In some embodiments, the transposome comprises a transposase. In some embodiments, the transposase is a Tn5 transposase, a Mu transposase, a Tn7 transposase, a Vibrio species transposase, or a functional derivative thereof. In particular embodiments, the transposase is a Tn5 transposase.

In some embodiments, the barcoded extension product comprises the transposon end sequence at a 3′ end of the barcoded extension product.

In some embodiments, the transposon end sequence comprises a sequencing primer sequence. In some embodiments, the sequencing primer sequence is a P7 sequence.

In some embodiments, the tagmenting in (c) is performed in a presence of a permeabilization agent. In some embodiments, the permeabilization agent comprises a detergent (Proteinase K, pepsin, or collagenase).

### A. Systems and Methods for Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion or a well. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more beads. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead.

Unique identifiers, such as barcodes, may be injected into the droplets previous to, subsequent to, or concurrently with droplet generation, such as via a bead, as described elsewhere herein.

The methods and systems of the present disclosure may comprise methods and systems for generating one or more partitions such as droplets. The droplets may comprise a plurality of droplets in an emulsion. In some examples, the droplets may comprise droplets in a colloid. In some cases, the emulsion may comprise a microemulsion or a nanoemulsion. In some examples, the droplets may be generated with aid of a microfluidic device and/or by subjecting a mixture of immiscible phases to agitation (e.g., in a container). In some cases, a combination of the mentioned methods may be used for droplet and/or emulsion formation.

The partitions described herein may comprise small volumes, for example, less than about 10 microliters (μL), 5 μL, 1 μL, 10 nanoliters (nL), 5 nL, 1 nL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet-based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with beads, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Patent Application No. PCT/US2020/17785 and U.S. Patent Application Publication No. US20220025438, which are entirely incorporated herein by reference for all purposes.

### B. Microfluidic Systems

In some embodiments, the method further comprises, prior to hybridizing and reverse transcribing the mRNA analyte, partitioning a cell, cell bead, or nucleus comprising the mRNA analyte with the nucleic acid barcode molecule attached to the solid support. In some embodiments, the partition comprises a well. In some embodiments, the partition comprises a droplet. In some embodiments, the partitioning utilizes a microfluidic device. Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particles in partitions, methods of increasing the throughput of droplet generation, and various geometries, architectures, and configurations of microfluidic devices and channels are described in U.S. Patent Application Publication Nos. 2019/0367997 and 2019/0064173, each of which is entirely incorporated herein by reference for all purposes.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as biological particles, in some cases, individual biological particles such as single cells. In some examples, reagents may be encapsulated and/or partitioned (e.g., co-partitioned with biological particles) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

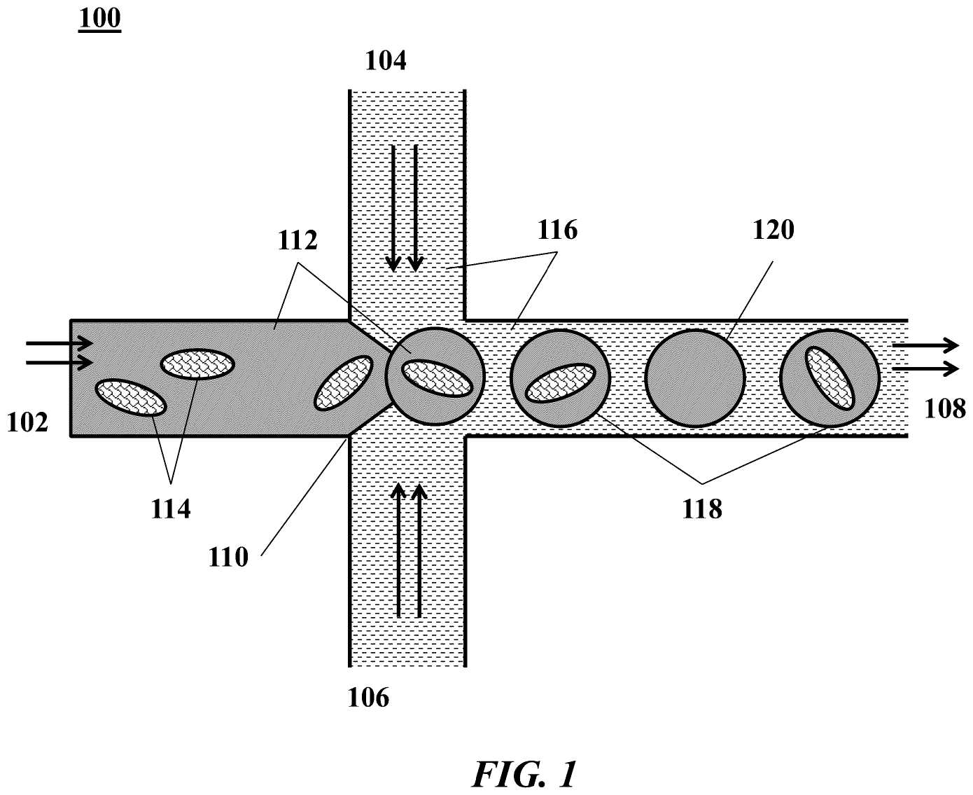

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, which includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometrics. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, flows can be controlled so as to present a non-Poissonian distribution of single-occupied partitions while providing lower levels of unoccupied partitions (e.g., no more than about 50%, about 25%, or about 10% unoccupied). The above noted ranges of unoccupied partitions can be achieved while still providing any of the single occupancy rates described above.

As will be appreciated, the above-described occupancy rates are also applicable to partitions that include both biological particles and additional reagents, such as beads (e.g., gel beads) carrying nucleic acid barcode molecules (e.g., oligonucleotides).

In some examples, a partition of the plurality of partitions may comprise a single biological particle (e.g., a single cell or a single nucleus of a cell). In some examples, a partition of the plurality of partitions may comprise multiple biological particles. Such partitions may be referred to as multiply occupied partitions, and may comprise, for example, two, three, four or more cells and/or beads (e.g., beads) comprising nucleic acid barcode molecules within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

Microfluidic systems for partitioning are further described in U.S. Patent Application Pub. No. US 2015/0376609, which is hereby incorporated by reference in its entirety.

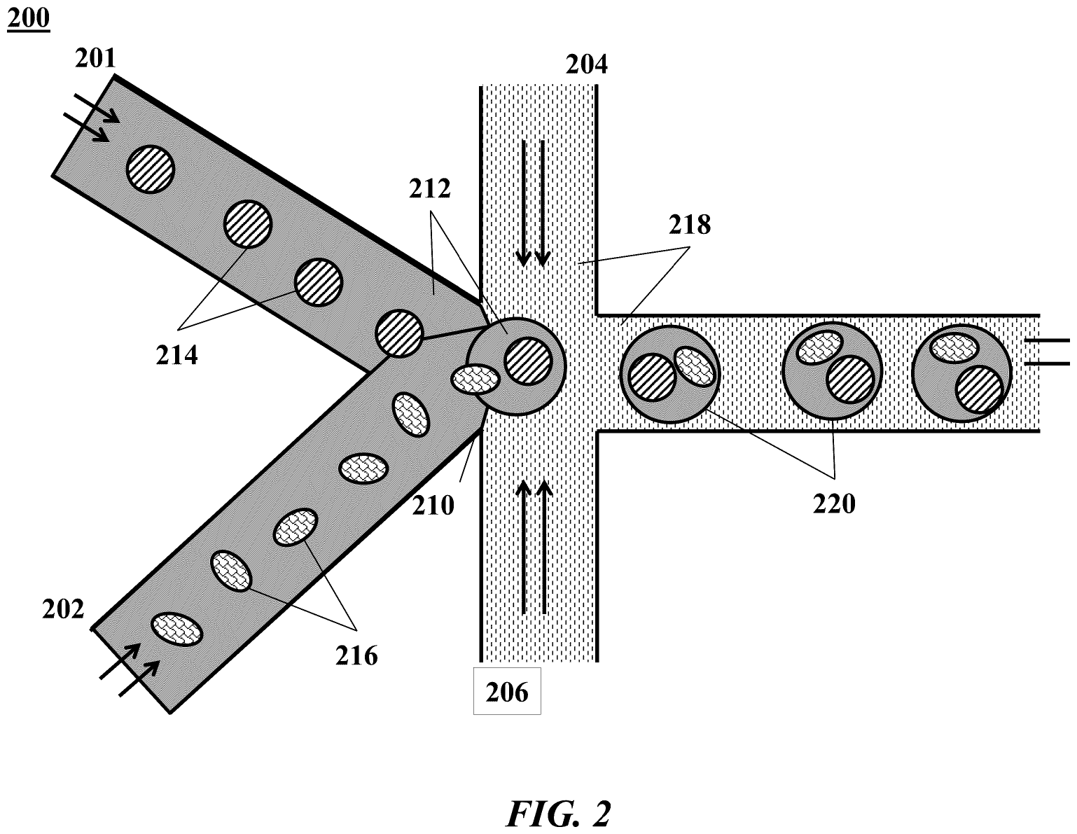

FIG. 2 shows an example of a microfluidic channel structure 200 for delivering barcode-carrying beads to droplets. The channel structure 200 can include channel segments 201, 202, 204, 206 and 208 communicating at a channel junction 210. In operation, the channel segment 201 may transport an aqueous fluid 212 that includes a plurality of beads 214 (e.g., with nucleic acid molecules, e.g., nucleic acid barcode molecules or barcoded oligonucleotides, molecular tags) along the channel segment 201 into junction 210. The plurality of beads 214 may be sourced from a suspension of beads. For example, the channel segment 201 may be connected to a reservoir comprising an aqueous suspension of beads 214. The channel segment 202 may transport the aqueous fluid 212 that includes a plurality of biological particles 216 along the channel segment 202 into junction 210. The plurality of biological particles 216 may be sourced from a suspension of biological particles. For example, the channel segment 202 may be connected to a reservoir comprising an aqueous suspension of biological particles 216. In some instances, the aqueous fluid 212 in either the first channel segment 201 or the second channel segment 202, or in both segments, can include one or more reagents, as further described below. A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 may deliver the discrete droplets to an outlet reservoir fluidly coupled to the channel segment 208, where they may be harvested. As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological particles in an alternating fashion, such that, for example, a droplet comprises a single bead and a single biological particle.

### C. Controlled Partitioning

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

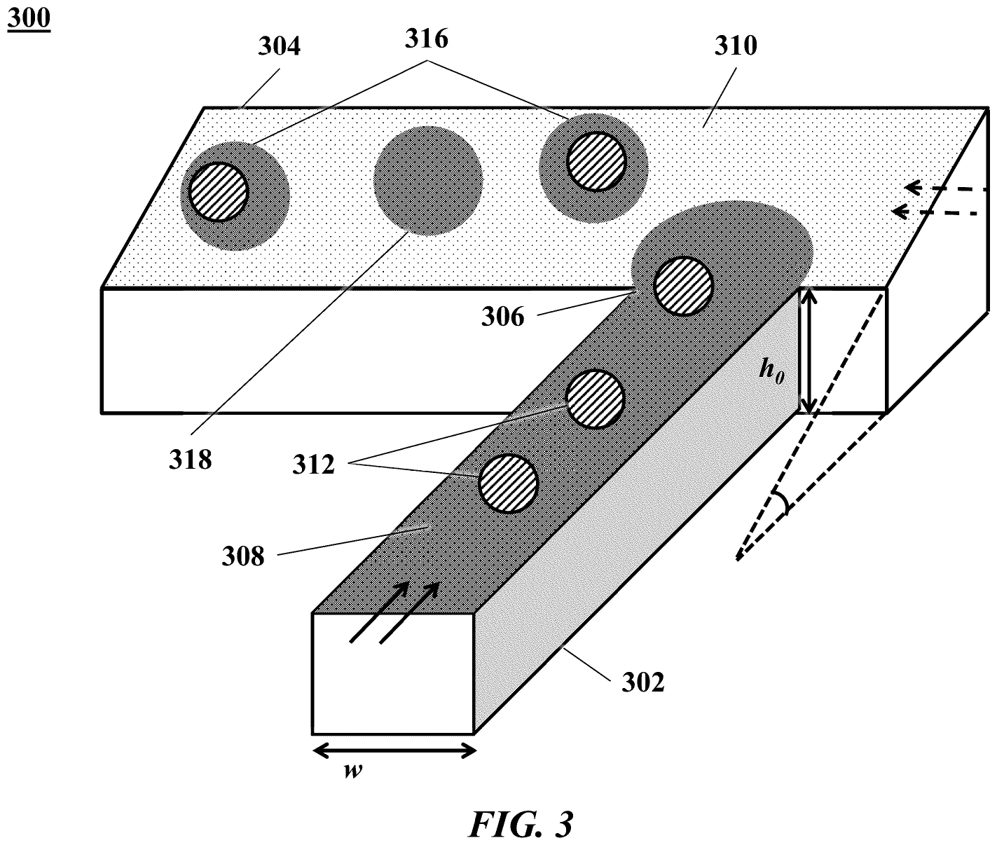

FIG. 3 shows an example of a microfluidic channel structure for the controlled partitioning of beads into discrete droplets. A channel structure 300 can include a channel segment 302 communicating at a channel junction 306 (or intersection) with a reservoir 304. The reservoir 304 can be a chamber. Any reference to “reservoir,” as used herein, can also refer to a “chamber.” In operation, an aqueous fluid 308 that includes suspended beads 212 may be transported along the channel segment 302 into the junction 306 to meet a second fluid 310 that is immiscible with the aqueous fluid 308 in the reservoir 304 to create droplets 316, 318 of the aqueous fluid 308 flowing into the reservoir 304. At the junction 306 where the aqueous fluid 308 and the second fluid 310 meet, droplets can form based on factors such as the hydrodynamic forces at the junction 306, flow rates of the two fluids 308, 310, fluid properties, and certain geometric parameters (e.g., w, h0, α, etc.) of the channel structure 300. A plurality of droplets can be collected in the reservoir 304 by continuously injecting the aqueous fluid 308 from the channel segment 302 through the junction 306.

In some instances, the aqueous fluid 308 can have a substantially uniform concentration or frequency of beads 312. The beads 312 can be introduced into the channel segment 302 from a separate channel (not shown in FIG. 3). The frequency of beads 312 in the channel segment 302 may be controlled by controlling the frequency in which the beads 312 are introduced into the channel segment 302 and/or the relative flow rates of the fluids in the channel segment 302 and the separate channel. In some instances, the beads can be introduced into the channel segment 30302 from a plurality of different channels, and the frequency controlled accordingly.

In some instances, the aqueous fluid 308 in the channel segment 302 can comprise biological particles. In some instances, the aqueous fluid 308 can have a substantially uniform concentration or frequency of biological particles. As with the beads, the biological particles can be introduced into the channel segment 302 from a separate channel. The frequency or concentration of the biological particles in the aqueous fluid 308 in the channel segment 302 may be controlled by controlling the frequency in which the biological particles are introduced into the channel segment 302 and/or the relative flow rates of the fluids in the channel segment 302 and the separate channel. In some instances, the biological particles can be introduced into the channel segment 302 from a plurality of different channels, and the frequency controlled accordingly. In some instances, a first separate channel can introduce beads and a second separate channel can introduce biological particles into the channel segment 302. The first separate channel introducing the beads may be upstream or downstream of the second separate channel introducing the biological particles.

The second fluid 310 can comprise an oil, such as a fluorinated oil, that includes a fluorosurfactant for stabilizing the resulting droplets, for example, inhibiting subsequent coalescence of the resulting droplets.

In some instances, the second fluid 310 may not be subjected to and/or directed to any flow in or out of the reservoir 304. For example, the second fluid 310 may be substantially stationary in the reservoir 304. In some instances, the second fluid 310 may be subjected to flow within the reservoir 304, but not in or out of the reservoir 304, such as via application of pressure to the reservoir 304 and/or as affected by the incoming flow of the aqueous fluid 308 at the junction 306. Alternatively, the second fluid 310 may be subjected and/or directed to flow in or out of the reservoir 304. For example, the reservoir 304 can be a channel directing the second fluid 310 from upstream to downstream, transporting the generated droplets.

Systems and methods for controlled partitioning are described further in International Patent Application No. PCT/US2018/047551 and U.S. Patent Application Publication No. US2020/0290048, which are hereby incorporated by reference in their entirety.

### D. Cell Beads

In another aspect, in addition to or as an alternative to droplet-based partitioning, biological particles (e.g., cells) may be comprised within (e.g., encapsulated within) a particulate material to form a “cell bead”. Methods and compositions drawn to cell beads and the like are described further in International Patent Application No. PCT/US2018/016019 and U.S. Patent Application No. US2018/0216162, which are hereby incorporated by reference in their entirety.

A cell bead can contain a biological particle (e.g., a cell) or macromolecular constituents (e.g., RNA, DNA, proteins, etc.) of a biological particle. A cell bead may include a single cell or multiple cells, or a derivative of the single cell or multiple cells. For example, after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

Cell beads can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli (or reagents).

Suitable polymers or gels may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. The conditions sufficient to polymerize or gel the precursors may comprise any conditions sufficient to polymerize or gel the precursors. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), electromagnetic radiation, mechanical stimuli, or any combination thereof.

In some cases, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 1, may be readily used in encapsulating biological particles (e.g., cells) as described herein. Exemplary methods for encapsulating biological particles (e.g., cells) are also further described in U.S. Patent Application Pub. No. US 2015/0376609 and International Patent Application No. PCT/US2018/016019, which are hereby incorporated by reference in their entirety. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the bead that includes the entrained biological particles. Examples of polymer precursor/initiator pairs include those described in U.S. Patent Application Publication No. 2014/0378345, which is entirely incorporated herein by reference for all purposes.

In some cases, encapsulated biological particles can be selectively releasable from the cell bead, such as through passage of time or upon application of a particular stimulus, that degrades the bead sufficiently to allow the biological particles (e.g., cell), or its other contents to be released from the bead, such as into a partition (e.g., droplet). Exemplary stimuli suitable for degradation of the bead are described in U.S. Patent Application Publication No. 2014/0378345, which is entirely incorporated herein by reference for all purposes.

The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The encapsulation of biological particles may constitute the partitioning of the biological particles into which other reagents are co-partitioned. Alternatively, or in addition, encapsulated biological particles may be readily deposited into other partitions (e.g., droplets) as described above.

### E. Solid Supports

Nucleic acid barcode molecules may be delivered to a partition (e.g., a droplet or well) via a solid support (e.g., a bead).

A solid support, e.g., a bead, may be porous, non-porous, hollow, solid, semi-solid, and/or a combination thereof. Beads may be solid, semi-solid, semi-fluidic, fluidic, and/or a combination thereof. In some instances, a solid support, e.g., a bead, may be at least partially dissolvable, disruptable, and/or degradable. In some cases, a solid support, e.g., a bead, may not be degradable. In some cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In some cases, the solid support, e.g., the bead, may be a silica bead. In some cases, the solid support, e.g., a bead, can be rigid. In other cases, the solid support, e.g., a bead, may be flexible and/or compressible.

In some embodiments, the solid support is magnetic, and the separating comprises magnetically separating. In some embodiments, the solid support comprises iron oxide. In some embodiments, the magnetic beads include iron oxide. In some embodiments, the magnetic beads include an iron oxide core. In some embodiments, the magnetic beads include an iron oxide core surrounded by a polymer coating. In some embodiments, the polymer coating includes polyethylene glycol (PEG), dextran, agarose, polyacrylamide, and/or silica. In particular embodiments, the polymer coating surrounding the magnetic core includes polyacrylamide.

In some embodiments, the barcode nucleic acid molecule comprises an affinity tag, and the separating comprises affinity purification. Examples of affinity purification methods include solid phase extraction, gel filtration, biotin (binding to streptavidin), streptavidin-binding peptide (SBP) purification, Glutathione S-transferase (GST) purification, and 6× hexahistidine-tag (His-tag) purification (using a nickel column). In particular embodiments, the affinity tag is biotin, and the affinity purification includes contacting with an additional solid support coated with streptavidin. In some embodiments, the solid support comprises an affinity tag, and the separating comprises affinity purification. In some embodiments, the solid support comprises a polymer of monomers and the nucleic acid barcode molecule is covalently attached to a monomer comprising the affinity tag.

In some embodiments, the solid support is a bead. In some embodiments, the solid support comprises polyacrylamide. Polyacrylamide polymers include crosslinked monomers. In some embodiments, the nucleic acid barcode molecule is covalently attached to a crosslinked monomer (e.g., an acrydite moiety). In some embodiments, the solid support include polyacrylamide, and an acrylamide monomer of the polyacrylamide is covalently attached to an affinity tag (e.g., biotin) and the nucleic acid barcode molecule.

In some embodiments, the solid support comprises an affinity tag, and the separating comprises affinity purification. In some embodiments, the support comprises a polymer of monomers and the nucleic acid barcode molecule is covalently attached to a monomer comprising the affinity tag.

In some embodiments, the bead includes a functional group on the surface. In some embodiments, the functional group is a moiety that allows affinity-based separation of the beads and associated nucleic acid barcode molecules from other nucleic acid molecules and/or reagents. In some embodiments, the functional group moiety is a protein. In some embodiments, the method includes separating the beads and associated nucleic acid barcode molecules by contacting the beads with an additional support including a ligand that specifically binds to the functional group protein on the beads. In some embodiments, the ligand is an antibody that specifically binds to the functional group protein. In some embodiments, the functional group protein is biotin and the ligand is streptavidin.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., via a nucleic acid barcode molecule), to a partition via any suitable mechanism. Nucleic acid barcode molecules can be delivered to a partition via a bead. Beads are described in further detail below.

In some cases, nucleic acid barcode molecules can be initially associated with the bead and then released from the bead. Release of the nucleic acid barcode molecules can be passive (e.g., by diffusion out of the bead). In addition, or alternatively, release from the bead can be upon application of a stimulus which allows the nucleic acid barcode molecules to dissociate or to be released from the bead. Such stimulus may disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof. Alternatively, the nucleic acid barcode molecule may lack a releasable attachment, such that the nucleic acid barcode remains attached to the bead during the course of the workflow. For example, the bead may be a crosslinked polymer bead that does not include labile bonds such as disulfide bonds linking monomers of the polymer. Additionally, the workflow may exclude a step of releasing the nucleic acid barcode molecule from the bead.

Methods and systems for partitioning barcode-carrying beads into droplets are provided herein, and in in US. Patent Publication Nos. 2019/0367997 and 2019/0064173, and International Patent Application No. PCT/US20/17785, each of which is herein entirely incorporated by reference for all purposes.

A bead may be porous, non-porous, solid, semi-solid, semi-fluidic, fluidic, and/or a combination thereof. In some instances, a bead may be dissolvable, disruptable, and/or degradable. Degradable beads, as well as methods for degrading beads, are described in International Patent Application Publication No. PCT/US2014/044398, which is hereby incorporated by reference in its entirety. In some cases, any combination of stimuli, e.g., stimuli described in PCT/US2014/044398 and US Patent Application Pub. No. 2015/0376609, hereby incorporated by reference in its entirety, may trigger degradation of a bead. For example, a change in pH may enable a chemical agent (e.g., DTT) to become an effective reducing agent. In other examples, a reducing agent (e.g., DTT) may be used to degrade the bead. In some aspects wherein the bead is degradable, nucleic acid barcode molecules may be covalently attached to monomers that may be re-crosslinked following the dissolving, disrupting, or degrading of the bead, to reform separatable particles to separate nucleic acid barcode molecules from other nucleic acid molecules and/or reagents.

In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. See, e.g., PCT/US2014/044398, which is hereby incorporated by reference in its entirety. Beads may also be formed from materials other than polymers, including lipids, micelles, ceramics, glass-ceramics, material composites, metals, other inorganic materials, and others.

In some cases, the bead may comprise covalent or ionic bonds between polymeric precursors (e.g., monomers, oligomers, linear polymers), nucleic acid barcode molecules (e.g., oligonucleotides), primers, and other entities. In some cases, the covalent bonds can be carbon-carbon bonds, thioether bonds, or carbon-heteroatom bonds.

In some cases, a plurality of nucleic acid barcode molecules may be attached to a bead. The nucleic acid barcode molecules may be attached directly or indirectly to the bead. In some cases, the nucleic acid barcode molecules may be covalently linked to the bead. In some cases, the nucleic acid barcode molecules are covalently linked to the bead via a linker. In some cases, the linker is a degradable linker. In some cases, the linker comprises a labile bond configured to release the nucleic acid barcode molecule of said plurality of nucleic acid barcode molecules. In some cases, the labile bond comprises a disulfide linkage.

Activation or disruption of disulfide linkages within a bead can be controlled such that only a small number of disulfide linkages are activated or disrupted. Methods of controlling activation of disulfide linkages within a bead are described in International Patent Application No. PCT/US2014/044398, which is hereby incorporated by reference in its entirety.

In some cases, a bead may comprise an acrydite moiety, which in certain aspects may be used to attach one or more nucleic acid barcode molecules (e.g., barcode sequence, nucleic acid barcode molecule, barcoded oligonucleotide, primer, or other oligonucleotide) to the bead. Acrydite moieties, as well as their uses in attaching nucleic acid molecules to beads, are described in PCT/US2014/044398, which is hereby incorporated by reference in its entirety.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule, e.g., a nucleic acid barcode molecule described herein.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. Exemplary precursors comprising functional groups are described in PCT/US2014/044398, which is hereby incorporated by reference in its entirety.

Other non-limiting examples of labile bonds that may be coupled to a precursor or bead are described in PCT/US2014/044398, which is hereby incorporated by reference in its entirety. A bond may be cleavable via other nucleic acid molecule targeting enzymes, such as restriction enzymes (e.g., restriction endonucleases), as described further below.

In some cases, a plurality of nucleic acid barcode molecules may be attached to a bead via non-covalent bonds. For example, the plurality of nucleic acid barcode molecules may be associated with a bead via an ionic interaction, electrostatic interactions, metallic bond, hydrogen bonding, van der Waals interactions, etc. In some cases, the non-covalent bond may be degraded upon application of a stimulus, e.g., a thermal, photo, magnetic, electrical, chemical stimulus (e.g., change in pH, ion concentration, etc.).

Species may be encapsulated in beads during bead generation (e.g., during polymerization of precursors). Such species may or may not participate in polymerization. See, e.g., PCT/US2014/044398, which is hereby incorporated by reference in its entirety. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Alternatively, or in addition, species may be partitioned in a partition (e.g., droplet) during or subsequent to partition formation. Such species may include, without limitation, the above-mentioned species that may also be encapsulated in a bead.

In some cases, beads can be non-covalently loaded with and/or coupled to one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

### F. Nucleic Acid Barcode Molecules for Single Cell Workflows

In some embodiments, the barcoded nucleic acid molecule is a partially double-stranded RNA/DNA molecule. In some embodiments, the partially double-stranded RNA/DNA molecule comprises an RNA sequence of the nucleic acid analyte and a DNA sequence complementary to the sequence of the nucleic acid analyte.

In some embodiments, the barcode sequence of the nucleic acid barcode molecule comprises a partition-specific barcode sequence.

In some embodiments, the nucleic acid barcode molecule further comprises one or more functional domains, a unique molecular identifier, or a combination thereof. In some embodiments, the one or more functional domains comprises a sequencing primer sequence.

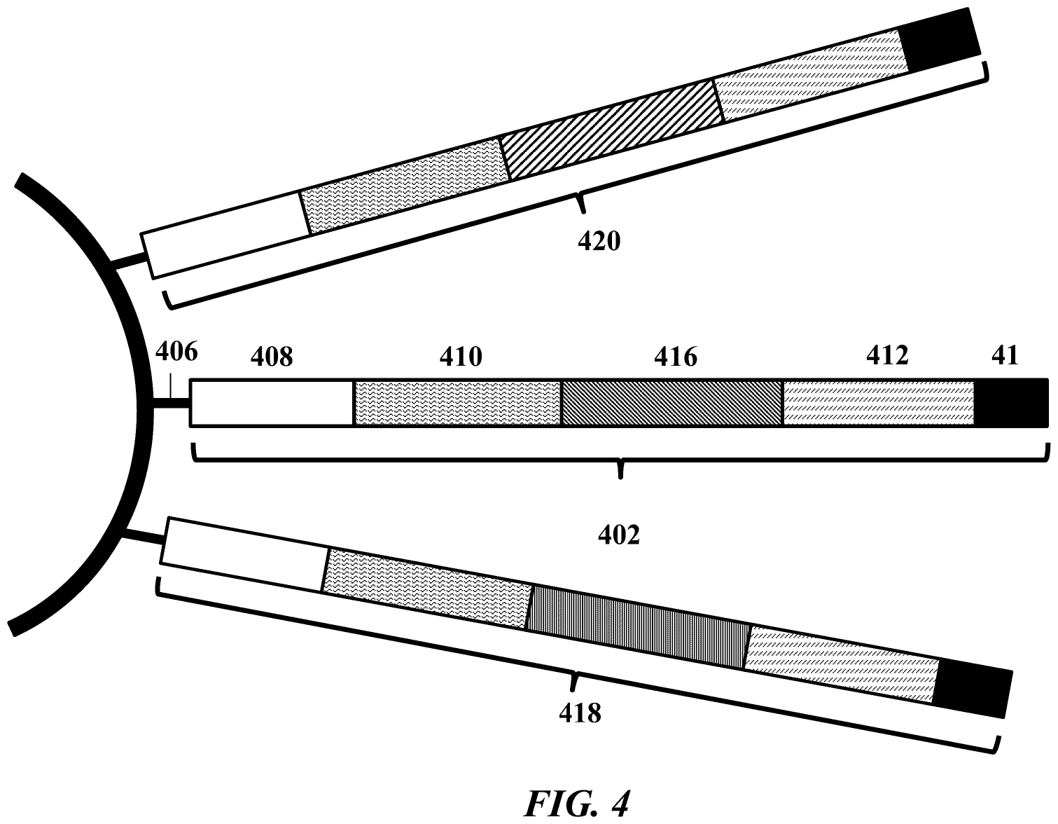

FIG. 4 illustrates an example of a barcode-carrying bead. A nucleic acid barcode molecule 402 can be coupled to a bead 404 by a linker 406, such as, for example, a disulfide linker. The same bead 404 may be coupled (e.g., via releasable linkage) to one or more other nucleic acid barcode molecules 418, 420. The nucleic acid barcode molecule 402 may be or comprise a barcode. As noted elsewhere herein, the structure of the barcode may comprise a number of sequence elements. The nucleic acid barcode molecule 402 may comprise a functional sequence 408 that may be used in subsequent processing. For example, the functional sequence 408 may include one or more of a sequencer specific flow cell attachment sequence (e.g., a P5 sequence for Illumina® sequencing systems) and a sequencing primer sequence (e.g., a RI primer for Illumina® sequencing systems), or partial sequence(s) thereof, and a region that is complementary to a region of an additional oligonucleotide used for generating an extended product for circularization. The nucleic acid barcode molecule 402 may comprise a barcode sequence 410 for use in barcoding the sample (e.g., DNA, RNA, protein, etc.). In some cases, the barcode sequence 410 can be bead-specific such that the barcode sequence 410 is common to all nucleic acid barcode molecules (e.g., including nucleic acid barcode molecule 402) coupled to the same bead 404. Alternatively, or in addition, the barcode sequence 410 can be partition-specific such that the barcode sequence 410 is common to all nucleic acid barcode molecules coupled to one or more beads that are partitioned into the same partition. The nucleic acid barcode molecule 402 may comprise sequence 412 complementary to an analyte of interest, e.g., a priming sequence. Sequence 412 can be a poly-T sequence complementary to a poly-A tail of an mRNA analyte, a targeted priming sequence, and/or a random priming sequence. The nucleic acid barcode molecule 402 may comprise an anchoring sequence 414 to ensure that the specific priming sequence 412 hybridizes at the sequence end (e.g., of the mRNA). For example, the anchoring sequence 414 can include a random short sequence of nucleotides, such as a 1-mer, 2-mer, 3-mer or longer sequence, which can ensure that a poly-T segment is more likely to hybridize at the sequence end of the poly-A tail of the mRNA. In some embodiments, the nucleic acid barcode molecule includes two barcode sequences separated by a primer region (including one or more primer binding sites).

The nucleic acid barcode molecule 402 may comprise a unique molecular identifying sequence 416 (e.g., unique molecular identifier (UMI)). In some cases, the unique molecular identifying sequence 416 may comprise from about 5 to about 8 nucleotides. Alternatively, the unique molecular identifying sequence 416 may compress less than about 5 or more than about 8 nucleotides. The unique molecular identifying sequence 416 may be a unique sequence that varies across individual nucleic acid barcode molecules (e.g., 402, 418, 420, etc.) coupled to a single bead (e.g., bead 404). In some cases, the unique molecular identifying sequence 416 may be a random sequence (e.g., such as a random N-mer sequence). For example, the UMI may provide a unique identifier of the starting analyte (e.g., mRNA) molecule that was captured, in order to allow quantitation of the number of original expressed RNA molecules. As will be appreciated, although FIG. 4 shows three nucleic acid barcode molecules 402, 418, 420 coupled to the surface of the bead 404, an individual bead may be coupled to any number of individual nucleic acid barcode molecules, for example, from one to tens to hundreds of thousands, millions, or even a billion of individual nucleic acid barcode molecules. The respective barcodes for the individual nucleic acid barcode molecules can comprise both common sequence segments or relatively common sequence segments (e.g., 408, 410, 412, etc.) and variable or unique sequence segments (e.g., 416) between different individual nucleic acid barcode molecules coupled to the same bead.

In operation, a biological particle (e.g., cell, DNA, RNA, etc.) can be co-partitioned along with a barcode bearing bead 404. The nucleic acid barcode molecules 402, 418, 420 can be released from the bead 404 in the partition. By way of example, in the context of analyzing sample RNA, the poly-T segment (e.g., 412) of one of the released nucleic acid barcode molecules (e.g., 402) can hybridize to the poly-A tail of a mRNA molecule. Reverse transcription may result in a cDNA transcript of the mRNA, but which transcript includes each of the sequence segments 408, 410, 416 of the nucleic acid barcode molecule 402. Because the nucleic acid barcode molecule 402 comprises an anchoring sequence 414, it will more likely hybridize to and prime reverse transcription at the sequence end of the poly-A tail of the mRNA. Within any given partition, all of the cDNA transcripts of the individual mRNA molecules may include a common barcode sequence segment 410. However, the transcripts made from the different mRNA molecules within a given partition may vary at the unique molecular identifying sequence 412 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid barcode molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise at least 2, 3, 4, 5, 6, 7, 8, 9, 10 or more different capture sequences or binding sequences configured to bind to different respective capture sequences or binding sequences, respectively. Alternatively, or in addition, a bead may comprise at most about 10, 9, 8, 7, 6, 5, 4, 3, or 2 different capture sequences or binding sequences configured to bind to different respective capture sequences or binding sequences. In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, an biological particle (e.g., cell, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules.

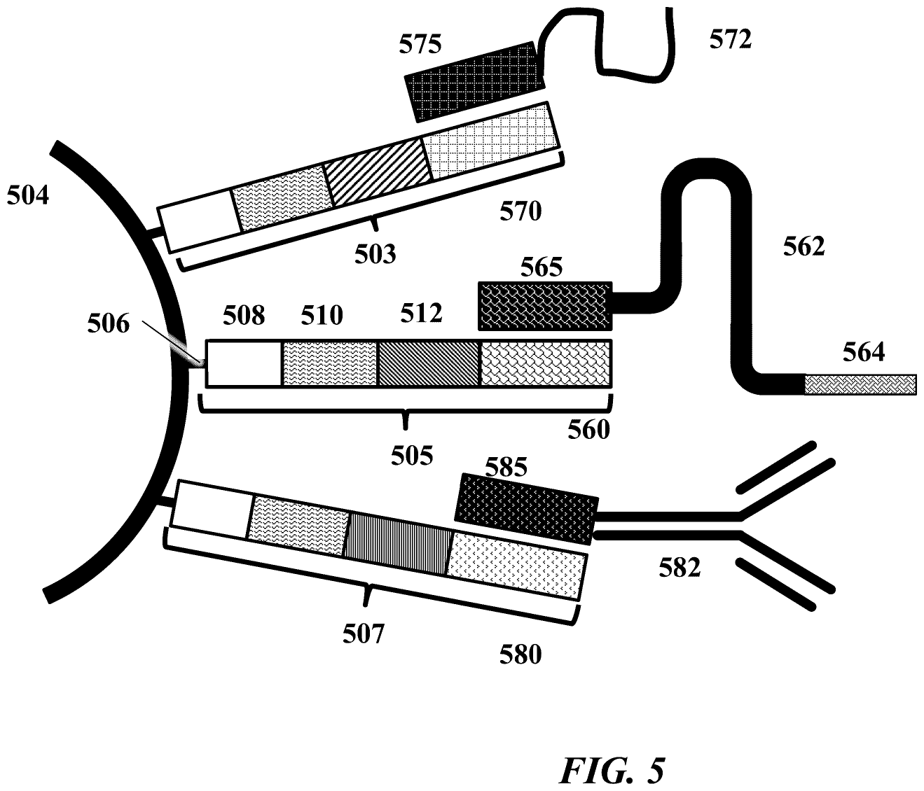

FIG. 5 illustrates an example of a barcode-carrying bead carrying additional nucleic acid barcode molecules. A nucleic acid barcode molecule 505, such as an oligonucleotide, can be coupled to a bead 504 by a linkage 506, such as, for example, a releasable linker. The nucleic acid barcode molecule 505 may comprise a first capture sequence 560. The same bead 504 may be coupled (e.g., via releasable linkage) to one or more other nucleic acid molecules 503, 507 comprising other capture sequences. The nucleic acid barcode molecule 505 may be or comprise a barcode. As noted elsewhere herein, the structure of the barcode may comprise a number of sequence elements, such as a functional sequence 508 (e.g., flow cell attachment sequence, sequencing primer sequence, region of complementarity with an additional oligonucleotide used for generating an extension product, etc.), a barcode sequence 510 (e.g., bead-specific sequence common to bead, partition-specific sequence common to partition, etc.), and a unique molecular identifier 512 (e.g., unique sequence within different molecules attached to the bead), or partial sequences thereof. The capture sequence 560 may be configured to attach to a corresponding capture sequence 565. In some instances, the corresponding capture sequence 565 may be coupled to another molecule that may be an analyte or an intermediary carrier. For example, as illustrated in FIG. 5, the corresponding capture sequence 565 is coupled to a guide RNA molecule 562 comprising a target sequence 564, wherein the target sequence 564 is configured to attach to the analyte. Another oligonucleotide molecule 507 attached to the bead 504 comprises a second capture sequence 580 which is configured to attach to a second corresponding capture sequence 585. As illustrated in FIG. 5, the second corresponding capture sequence 585 is coupled to an antibody 582. In some cases, the antibody 582 may have binding specificity to an analyte (e.g., surface protein). Alternatively, the antibody 582 may not have binding specificity. Another oligonucleotide molecule 503 attached to the bead 504 comprises a third capture sequence 570 which is configured to attach to a third corresponding capture sequence 575. As illustrated in FIG. 5, the third corresponding capture sequence 575 is coupled to a molecule 572. The molecule 572 may or may not be configured to target an analyte. The other oligonucleotide molecules 503, 507 may comprise the other sequences (e.g., functional sequence, barcode sequence, UMI, etc.) described with respect to oligonucleotide molecule 505. While a single oligonucleotide molecule comprising each capture sequence is illustrated in FIG. 5, it will be appreciated that, for each capture sequence, the bead may comprise a set of one or more oligonucleotide molecules each comprising the capture sequence. For example, the bead may comprise any number of sets of one or more different capture sequences. Alternatively, or in addition, the bead 504 may comprise other capture sequences. Alternatively, or in addition, the bead 504 may comprise fewer types of capture sequences (e.g., two capture sequences). Alternatively, or in addition, the bead 504 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression. In some embodiments, two barcode sequences of the nucleic acid barcode molecule are separated by a primer region (e.g., including at least one priming sequence).

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. In some embodiments, the bead is composed of a polymer of crosslinked monomers, and upon degradation of the polymer, monomers covalently attached to the nucleic acid barcode molecule are released. In such embodiments, the monomer attached to the nucleic acid barcode molecule includes a feature (e.g., an affinity tag) that can be used for capture, to separate the nucleic acid barcode molecule from other nucleic acid molecules following a workflow step (e.g., following tagmenting). Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. See, e.g., PCT/US2014/044398, which is hereby incorporated by reference in its entirety.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. Sec, e.g., PCT/US2014/044398, which is hereby incorporated by reference in its entirety.

As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full RI sequence, and/or partial RI sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences (e.g., restriction sites, transposition sites). Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, beads are provided that include large numbers of the above-described nucleic acid barcode molecules releasably attached to the beads, where all or at least a subset of the nucleic acid barcode molecules attached to a particular bead will include a common nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid barcode molecules into the partitions, as they are capable of carrying large numbers of nucleic acid barcode molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, beads can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. In some embodiments, the number of nucleic acid molecules including the barcode sequence on an individual bead is between about 1,000 to about 10,000 nucleic acid molecules, about 5,000 to about 50,000 nucleic acid molecules, about 10,000 to about 100,000 nucleic acid molecules, about 50,000 to about 1,000,000 nucleic acid molecules, about 100,000 to about 10,000,000 nucleic acid molecules, about 1,000,000 to about 1 billion nucleic acid molecules.

Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acids, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules.

In some cases, the resulting population of partitions provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences. Additionally, each partition of the population can include between about 1,000 to about 10,000 nucleic acid barcode molecules, about 5,000 to about 50,000 nucleic acid barcode molecules, about 10,000 to about 100,000 nucleic acid barcode molecules, about 50,000 to about 1,000,000 nucleic acid barcode molecules, about 100,000 to about 10,000,000 nucleic acid barcode molecules, about 1,000,000 to about 1 billion nucleic acid barcode molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads or otherwise results in release of the nucleic acid molecules from the beads. In one case, such compositions include the polyacrylamide matrices described above for encapsulation of biological particles and may be degraded for release of the attached nucleic acid molecules through exposure to a reducing agent, such as DTT.

### G. Single Cell Workflow Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

The methods and systems of the present disclosure may comprise microfluidic devices and methods of use thereof, which may be used for co-partitioning biological particles with reagents. Such systems and methods are described in U.S. Patent Publication No. US/20190367997, which is herein incorporated by reference in its entirety for all purposes.

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments of the microfluidic devices described elsewhere herein may be coupled to any of a variety of different fluid sources or receiving components, including reservoirs, tubing, manifolds, or fluidic components of other systems. As will be appreciated, the microfluidic channel structures may have various geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion-based partitioning such as encapsulation of biological particles that may be in addition to or in place of droplet partitioning, where any pore size of the encapsulate is sufficiently small to retain nucleic acid fragments of a given size, following cellular disruption.

Alternatively, or in addition to, the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., a cell or a nucleus in a polymer matrix), the biological particles may be exposed to an appropriate stimulus to release the biological particles or their contents from a co-partitioned bead. For example, in some cases, a chemical stimulus may be co-partitioned along with an encapsulated biological particle to allow for the degradation of the bead and release of the cell or its contents into the larger partition. In some cases, this stimulus may be the same as the stimulus described elsewhere herein for release of nucleic acid molecules (e.g., oligonucleotides) from their respective bead. In alternative examples, this may be a different and non-overlapping stimulus, in order to allow an encapsulated biological particle to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

Additional reagents may also be co-partitioned with the biological particle, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, restriction enzymes, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some cases, template switching can be used to increase the length of a cDNA. In some cases, template switching can be used to append a predefined nucleic acid sequence to the cDNA. Template switching is further described in International Patent Application No. PCT/US2017/068320 and U.S. Pat. No. 10,011,872, which are hereby incorporated by reference in their entirety. Template switching oligonucleotides may comprise a hybridization region and a template region. Template switching oligonucleotides are further described in PCT/US2017/068320 and U.S. Pat. No. 10,011,872 which are hereby incorporated by reference in their entirety.

Any of the reagents described in this disclosure may be encapsulated in, or otherwise coupled to, a droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, and oligonucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles. In some aspects, this is performed by co-partitioning the individual biological particle or groups of biological particles with the unique identifiers, such as described above (with reference to FIG. 1 or 2).

In some cases, additional beads can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of beads from each source, while ensuring a given pairing or combination of such beads into a partition with a given number of biological particles (e.g., one biological particle and one bead per partition).

In some embodiments, following the generation of barcoded nucleic acid molecules according to methods disclosed herein, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken, and the contents of the droplet pooled for additional operations.

### H. Wells

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). In some embodiments, a well of a fluidic device is fluidically connected to another well of the fluidic device. Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

A well may comprise free reagents and/or reagents encapsulated in, or otherwise coupled to or associated with, beads, beads, or droplets.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In some cases, a well comprises a bead, or droplet that comprises a set of reagents that has a similar attribute (e.g., a set of enzymes, a set of minerals, a set of oligonucleotides, a mixture of different barcode molecules, a mixture of identical barcode molecules). In other cases, a bead or droplet comprises a heterogeneous mixture of reagents. In some cases, the heterogeneous mixture of reagents can comprise all components necessary to perform a reaction. In some cases, such mixture can comprise all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

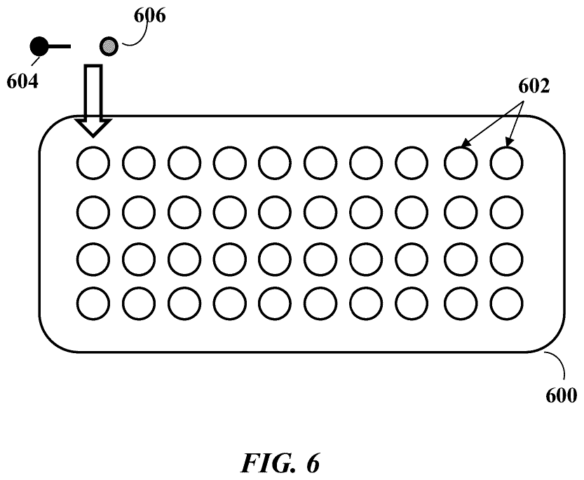

FIG. 6 schematically illustrates an example of a microwell array. The array can be contained within a substrate 600. The substrate 600 comprises a plurality of wells 602. The wells 602 may be of any size or shape, and the spacing between the wells, the number of wells per substrate, as well as the density of the wells on the substrate 600 can be modified, depending on the particular application. In one such example application, a sample molecule 606, which may comprise a cell or cellular components (e.g., nucleic acid molecules) is co-partitioned with a bead 604, which may comprise a nucleic acid barcode molecule coupled thereto. The wells 602 may be loaded using gravity or other loading technique (e.g., centrifugation, liquid handler, acoustic loading, optoelectronic, etc.). In some instances, at least one of the wells 602 contains a single sample molecule 606 (e.g., cell) and a single bead 604.

Reagents may be loaded into a well either sequentially or concurrently. In some cases, reagents are introduced to the device either before or after a particular operation. In some cases, reagents (which may be provided, in certain instances, in droplets, or beads) are introduced sequentially such that different reactions or operations occur at different steps. The reagents (or droplets, or beads) may also be loaded at operations interspersed with a reaction or operation step. For example, beads (or droplets) comprising reagents for fragmenting polynucleotides (e.g., restriction enzymes) and/or other enzymes (e.g., transposases, ligases, polymerases, etc.) may be loaded into the well or plurality of wells, followed by loading of droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead, or droplet. These beads, or droplets may be loaded into a partition (e.g., a microwell) before, after, or concurrently with the loading of a cell, such that each cell is contacted with a different bead, or droplet. This technique may be used to attach a unique nucleic acid barcode molecule to nucleic acid molecules obtained from each cell. Alternatively, or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in some instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or bead may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecules may be attached to a droplet or bead that has been partitioned into the well. The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. In some instances, the nucleic acid barcode molecules may be releasable from the bead or droplet. For instance, the nucleic acid barcode molecules may comprise a chemical cross-linker which may be cleaved upon application of a stimulus (e.g., photo-, magnetic, chemical, biological, stimulus). The nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In some instances nucleic acid barcode molecules attached to a bead in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to dynamic live-cell tracking, cell-cell interactions (when two or more cells are co-partitioned), cell proliferation, etc. Alternatively, or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized (e.g, using Proteinase K, pepsin, or collagenase) in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

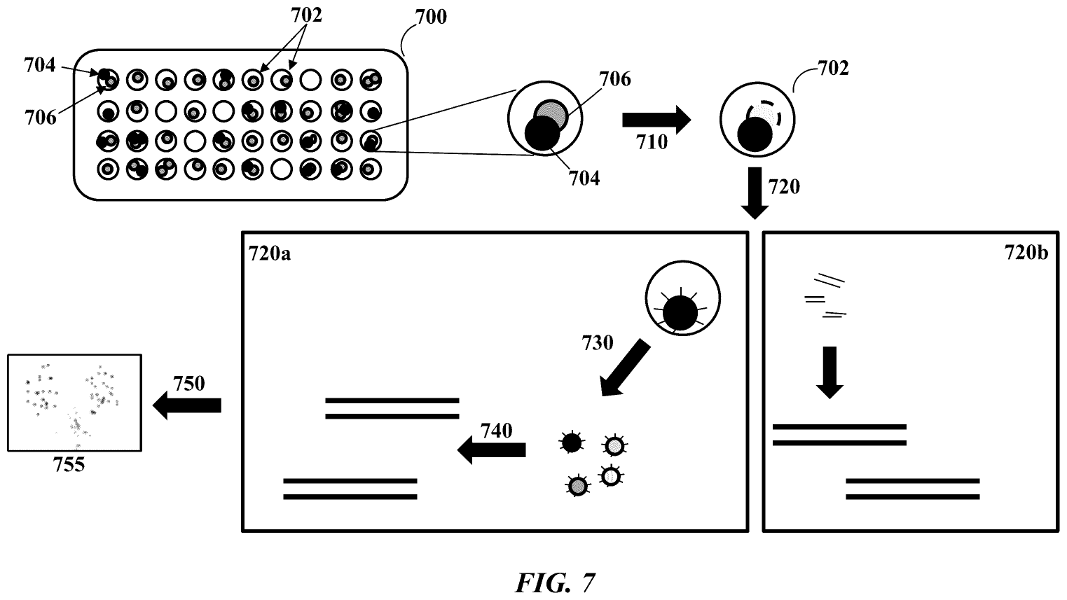

FIG. 7 schematically shows an example workflow for processing nucleic acid molecules within a sample. A substrate 700 comprising a plurality of microwells 702 may be provided. A sample 706 which may comprise a cell, cell bead, cellular components or analytes (e.g., proteins and/or nucleic acid molecules) can be co-partitioned, in a plurality of microwells 702, with a plurality of beads 704 comprising nucleic acid barcode molecules. During process 710, the sample 706 may be processed within the partition. For instance, in the case of live cells, the cell may be subjected to conditions sufficient to lyse the cells and release the analytes contained therein. In process 720, the bead 704 may be further processed. By way of example, processes 720a and 720b schematically illustrate different workflows, depending on the properties of the bead 704.

In 720a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachments may occur on the bead. In process 730, the beads 704 from multiple wells 702 may be collected and pooled. Further processing may be performed in process 740. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 750, further characterization, such as sequencing may be performed to generate sequencing reads. The sequencing reads may yield information on individual cells or populations of cells, which may be represented visually or graphically, e.g., in a plot 755.

In 720b, the bead comprises nucleic acid barcode molecules that are releasably attached thereto, as described below. The bead may degrade or otherwise release the nucleic acid barcode molecules into the well 702; the nucleic acid barcode molecules may then be used to barcode nucleic acid molecules within the well 702. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 750, further characterization, such as sequencing may be performed to generate sequencing reads. The sequencing reads may yield information on individual cells or populations of cells, which may be represented visually or graphically, e.g., in a plot 755.

### 1. Targeting GEX

The methods provided herein may comprise the use of a targeting process to, e.g., enrich selected nucleic acid molecules within a sample.

An exemplary target enrichment method may comprise providing a plurality of barcoded nucleic acid molecules and hybridizing barcoded nucleic acid molecules comprising targeted regions of interest to oligonucleotide probes (“baits”) which are complementary to the targeted regions of interest (or to regions near or adjacent to the targeted regions of interest). Baits may be attached to a capture molecule, including without limitation a biotin molecule. The capture molecule (e.g., biotin) can be used to selectively pull down the targeted regions of interest (for example, with magnetic streptavidin beads) to thereby enrich the resultant population of barcoded nucleic acid molecules for those containing the targeted regions of interest.

Another exemplary enrichment method may comprise providing a plurality of barcoded nucleic acid molecules comprising a plurality of different barcode sequences, identifying a barcode sequence of the plurality of different barcode sequences, and enriching barcoded nucleic acid molecules comprising the barcode sequence. Enriching may comprise performing a nucleic acid extension reaction using a barcoded nucleic acid molecule comprising the barcode sequence and a primer comprising a sequence specific for the barcode sequence to generate an enriched plurality of barcoded nucleic acid molecules comprising the barcode sequence of interest. Details of such processes and additional schemes are included in, for example, International Patent Application No. PCT/US2020/012413, U.S. Patent Application Publication No. US2022/0025435, and U.S. Pat. No. 11,000,049, and herein entirely incorporated by reference for all purposes.

The present disclosure provides methods and systems for multiplexing, and otherwise increasing throughput in, analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cell features may be used to characterize biological particles and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

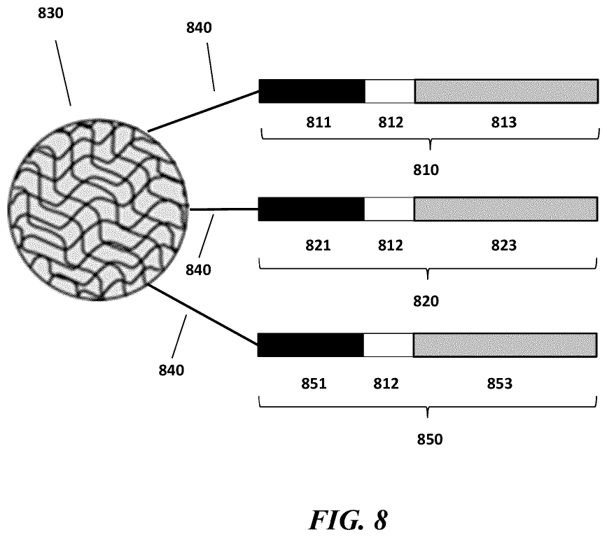

FIG. 8 illustrates another example of a barcode-carrying bead. In some embodiments, analysis of multiple analytes (e.g., RNA and one or more analytes using labelling agents described herein) may comprise nucleic acid barcode molecules as generally depicted in FIG. 8. In some embodiments, nucleic acid barcode molecules 910 and 920 are attached to support 930 via a releasable linkage 840 (e.g., comprising a labile bond) as described elsewhere herein. Nucleic acid barcode molecule 810 may comprise adapter sequence 811, barcode sequence 812 and capture sequence 813, and optionally a self-complementary region (not shown). Nucleic acid barcode molecule 820 may comprise adapter sequence 821, barcode sequence 812, and capture sequence 823, and optionally a self-complementary region (not shown), wherein capture sequence 823 comprises a different sequence than capture sequence 813. In some instances, adapter 811 and adapter 821 comprise the same sequence. In some instances, adapter 811 and adapter 821 comprise different sequences. Although support 830 is shown comprising nucleic acid barcode molecules 810 and 820, any suitable number of barcode molecules comprising common barcode sequence 812 are contemplated herein. For example, in some embodiments, support 830 further comprises nucleic acid barcode molecule 850. Nucleic acid barcode molecule 850 may comprise adapter sequence 851, barcode sequence 812 and capture sequence 853, and optionally a self-complementary region (not shown), wherein capture sequence 853 comprises a different sequence than capture sequence 813 and 823. In some instances, nucleic acid barcode molecules (e.g., 810, 820, 850) comprise one or more additional functional sequences, such as a UMI or other sequences described herein. The nucleic acid barcode molecules 810, 820 or 850 may interact with analytes as described elsewhere herein.

In some embodiments, biological particles (e.g., cells, nuclei) from a plurality of samples (e.g., a plurality of subjects) can be pooled, sequenced, and demultiplexed by identifying mutational profiles associated with individual samples and mapping sequence data from single biological particles to their source based on their mutational profile. See, e.g., Xu J. et al., Genome Biology Vol. 20, 290 (2019); Huang Y. et al., Genome Biology Vol. 20, 273 (2019); and Heaton et al., Nature Methods volume 17, pages 615-620(2020).

Gene expression data can reflect the underlying genome and mutations and structural variants therein. As a result, the variation inherent in the captured and sequenced RNA molecules can be used to identify genotypes de novo or used to assign molecules to genotypes that were known a priori. In some embodiments, allelic variation that is present due to haplotypic states (including linkage disequilibrium of the human leucocyte antigen loci (HLA), immune receptor loci (BCR), and other highly polymorphic regions of the genome), can also be used for demultiplexing. Expressed B cell receptors can be used to infer germline alleles from unrelated individuals, which information may be used for demultiplexing.

### III. Systems for Tagmentation-Based Single Cell Sequencing Assays

In addition to the methods described herein, the present disclosure also features tagmentation systems for the single cell detection of mRNA analytes from a biological sample. Thus, provided herein is a system including: (a) an mRNA analyte; (b) a nucleic acid barcode molecule comprising: (i) a barcode sequence and (ii) a domain that hybridizes to a 3′ sequence of the mRNA analyte, wherein the nucleic acid barcode molecule is attached to a solid support; and (c) a transposome comprising a transposase and a transposon end sequence, wherein the transposon end sequence comprises a sequencing primer sequence. In some embodiments, the components of the system are present in a partition (e.g., a well or droplet). In some embodiments, the solid support comprises a bead. In some embodiments, the solid support comprises a hydrogel. In some embodiments, the transposase is a Tn5 transposase, a Mu transposase, a Tn7 transposase, a Vibrio species transposase, or a functional derivative thereof. In some embodiments, the transposase is Tn5 transposase. In some embodiments, the transposome comprises two transposon end nucleic acid molecules. In some embodiments, the two transposon end nucleic acid molecule each comprises a sequencing primer sequence. In some embodiments, the nucleic acid barcode molecule includes a first sequencing primer (or part of a first sequencing primer) and the transposon end nucleic acid molecules include a second sequencing primer (or part of a second sequencing primer). In some embodiments, the system further includes a reverse transcriptase. In some embodiments, the system further includes a DNA polymerase. In some embodiments, the system further includes a plurality of dNTPs. In some embodiments, the system further includes an RNase. In some embodiments, the system further includes a DNase.

### IV. Kits for Tagmentation-Based Single Cell Sequencing Assays

In addition to the methods and compositions described herein, the present disclosure also features kits for single cell resolution of target analytes from a biological sample. Thus, provided herein are kits including: (a) nucleic acid barcode molecule comprising: (i) a barcode sequence and (ii) a domain that hybridizes to an mRNA analyte, wherein the nucleic acid barcode molecule is attached to a solid support; (b) a reverse transcriptase; (c) a transposome comprising a transposase and a transposon end sequence, wherein the transposon end sequence comprises a sequencing primer sequence; and (d) instructions for performing a method as provided herein. In some embodiments, the solid support comprises a hydrogel. In some embodiments, the system further comprises solid support comprises a bead. In some embodiments, the transposase is a Tn5 transposase, a Mu transposase, a Tn7 transposase, a Vibrio species transposase, or a functional derivative thereof. In some embodiments, the transposome comprises two transposon end sequences. In some embodiments, the kit comprises the reverse transcriptase. In some embodiments, the kit comprises the polymerase. In some embodiments, the polymerase is a DNA polymerase. In some embodiments, the kit further comprises a plurality of dNTPs. In some embodiments, the kit further comprises one or more permeabilization reagents. In some embodiments, the one or more permeabilization reagents comprises a protease, a surfactant, or a detergent. In some embodiments, the kit further comprises an RNase. In some embodiments, the kit further comprises a DNase. In some embodiments, the domain that hybridizes to an mRNA analyte comprises a polyT sequence.

### EXAMPLES

### Example 1—Tagmentation and Bead-Based Purification of Double-Stranded RNA/cDNA Hybrid Extension Products for Single Cell Sequencing

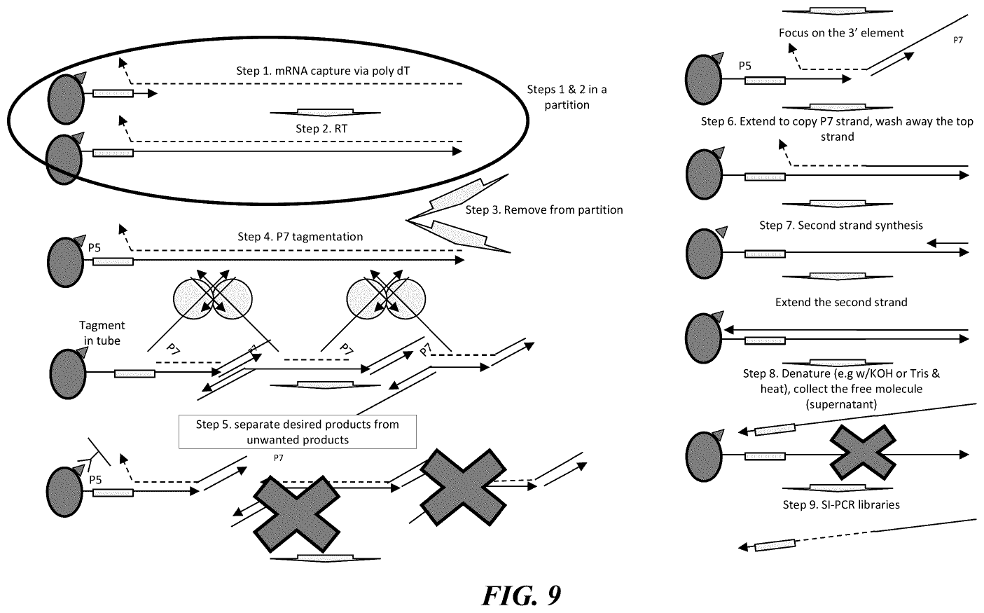

FIG. 9 is a schematic of a streamlined 3′ single cell sequencing library preparation workflow that can bypass various processes of a typical 3′ single sell sequencing library preparation workflow and avoid wasting reagents on unwanted fragments of the mRNA analyte. This method involves, in a partition, hybridizing a polyadenylated mRNA analyte from a biological sample using nucleic acid barcode molecule attached to a support and having a poly(T) sequence (FIG. 9, step 1). The support includes a plurality of nucleic acid barcode molecules, each sharing a same partition-specific barcode. The support also includes a functional group for downstream enrichment (e.g., biotin covalently attached to the bead).

Following mRNA hybridization, the nucleic acid barcode molecule is extended using a reverse transcriptase and using the mRNA as a template, thereby generating a cDNA molecule that includes both the barcode sequence and a sequence complementary to a sequence of the mRNA analyte (FIG. 9, step 2). The partially double-stranded RNA/cDNA is subjected to tagmentation by contacting with a transposome complex that includes a transposase and one or more transposon end or mosaic end (ME) sequences (FIG. 9, step 4). The one or more transposon end sequences includes a sequencing primer sequence, e.g., a P7 sequence, as shown. The transposase facilitates fragmentation and insertion of one or more transposon end sequences to the partially double-stranded RNA/cDNA.

Following tagmentation, the tagmented 3′ end fragment including (a) the original nucleic acid barcode molecule attached to the support and (b) a 3′ portion of the mRNA analyte is separated from unwanted tagmented fragments by enriching for the support (e.g., using streptavidin-coated beads when biotin is included in the support, or using a magnet when using magnetic beads). The tagmented 3′ end fragment is subsequently processed for library construction. The strand including the barcode sequence and copy of the mRNA analyte is extended to also include the transposon end sequence, and the strand including a portion of the mRNA analyte is then washed away, e.g., by treating with a base (e.g., KOH) or an RNase (FIG. 9, step 6). Next, a second strand primer is hybridized to the barcoded cDNA via the P7 sequence and extended for second strand synthesis, thereby generating a partially double-stranded cDNA molecule (FIG. 9, step 7). The newly synthesized second strand, which includes the complement of the barcode sequence is denatured and separated from the template strand (FIG. 9, step 8). The collected second strand cDNA product is next amplified for SI-PCR library construction (FIG. 9, step 9).

### Example 2—Tagmentation and Bead-Based Purification of Double-Stranded RNA/DNA Extension Products for Single Cell Sequencing of Single-Stranded RNA/DNA Hybrid Products

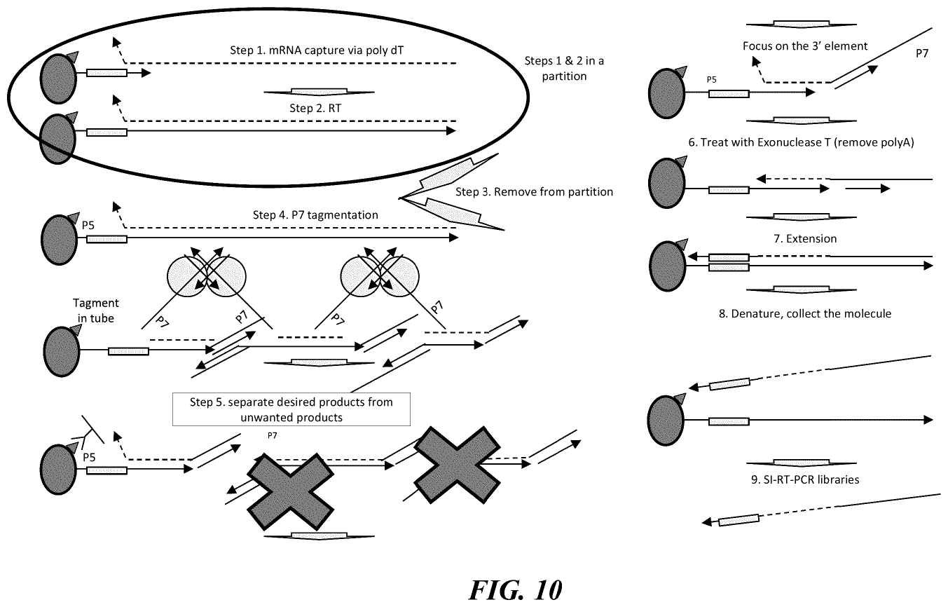

FIG. 10 shows another streamlined single cell sequencing library preparation workflow herein that can bypass various processes of a typical 3′ single sell sequencing library preparation workflow and avoid wasting reagents on unwanted fragments of the mRNA analyte. As previously described, a polyadenylated mRNA analyte from a biological sample is hybridized to a nucleic acid barcode molecule attached to a support and having a poly(T) sequence.

Steps 1-4 are conducted in the same manner as described for FIG. 9.

Following tagmentation, the tagmented 3′ end fragment including (a) the original nucleic acid barcode molecule attached to the support and (b) a 3′ portion of the mRNA analyte is separated from unwanted tagmented fragments by enriching for the support (e.g., using streptavidin-coated beads when biotin is included in the support, or using a magnet when using magnetic beads). The tagmented 3′ end fragment (“the 3′ element” as shown in FIG. 10) is subsequently processed for library construction. Distinct from the workflow of FIG. 10, the barcoded nucleic acid fragment is processed using an exonuclease, e.g., exonuclease T, to remove 3′ polyadenylated overhang of the mRNA analyte that is not hybridized (i.e., non-complementary), thereby generating a 3′ end available for extension. The tagmented 3′ end fragment of the mRNA analyte is then extended to include a complement of the sequence of the nucleic acid barcode molecule (FIG. 10, step 7). Instead of washing away the strand including a portion of the mRNA analyte, the nucleic acid barcode molecule attached to the support is extracted and the supernatant including the desired strand is retained. The collected RNA/cDNA hybrid strand is then amplified for sample indexing-reverse transcription-PCR (SI-RT-PCR) library construction.

What is claimed is:

1. A method for processing an mRNA molecule, the method comprising:
(a) in a partition, hybridizing the mRNA molecule to a barcode nucleic acid molecule attached to a solid support, wherein the barcode nucleic acid molecule comprises: (i) a barcode sequence and (ii) a domain that hybridizes to a 3′ sequence of the mRNA molecule;
(b) extending the barcode nucleic acid molecule, thereby generating a barcoded nucleic acid molecule annealed to the mRNA molecule, wherein the barcoded nucleic acid molecule comprises a sequence complementary to the mRNA molecule;
(c) tagmenting the barcoded nucleic acid molecule annealed to the mRNA molecule, thereby generating a plurality of tagmented fragments comprising a first tagmented fragment, wherein the first tagmented fragment comprises a first strand comprising an mRNA fragment comprising 3′ sequence of the mRNA molecule covalently attached to the transposon end sequence, and a second strand comprising the barcode nucleic acid molecule and a portion of the sequence complementary to the mRNA molecule; and
(d) separating the first tagmented fragment from other tagmented fragments of the plurality of tagmented fragments.

2. The method of claim 1, wherein steps (c) and (d) are performed outside of the partition.

3. The method of claim 1, wherein steps (b), (c), and (d) are performed outside the partition.

4. The method of claim 1, further comprising, after the tagmenting in (c), extending the second strand using the first strand as a template, thereby generating an extended barcoded fragment comprising: (i) the barcode sequence and (ii) a copy of the mRNA fragment, and (iii) the transposon end sequence.

5. The method of claim 4, wherein the extending the second strand is after the separating in (d).

6. The method of claim 1, wherein the solid support is magnetic, and the separating comprises magnetically separating.

7. The method of claim 6, wherein the solid support comprises iron oxide.

8. The method of claim 1, wherein the barcode nucleic acid molecule comprises an affinity tag, and the separating comprises affinity purification.

9. The method of claim 1, wherein the solid support comprises an affinity tag, and the separating comprises affinity purification.

10. The method of claim 1, wherein the solid support is a bead.

11. The method of claim 1, wherein the solid support comprises polyacrylamide.

12. The method of claim 4, further comprising: (i) hybridizing a primer to the extended barcoded fragment, and (ii) extending the primer, thereby generating a copy of the extended barcoded fragment.

13. The method of claim 12, further comprising separating the copy of the extended barcoded fragment from the extended barcode fragment.

14. The method of claim 12, wherein the primer comprises a sequence complementary to the transposon end sequence.

15. The method of claim 12, further comprising determining a sequence of the extended barcoded fragment.

16. The method of claim 12, further comprising sequencing the extended barcoded fragment.

17. The method of claim 4, wherein following the (a) hybridizing, a terminal end of 3′ sequence of the mRNA molecule is not hybridized to the barcode nucleic acid molecule and after the tagmenting in (c), the method further comprises removing the terminal end of 3′ sequence that is not hybridized.

18. The method of claim 17, wherein the terminal end is a terminal portion of a poly-A tail.

19. The method of claim 17, wherein the removing comprises treating with a nuclease.

20. The method of claim 17, further comprising, after the removing, extending the tagmented fragment using the barcoded nucleic acid molecule as a template, thereby generating a barcoded extension product comprising: (i) the barcode sequence or a complement thereof, and (ii) a copy of the tagmented fragment.

21. The method of claim 20, further comprising determining a sequence of the barcoded extension product or an intermediate thereof.

22. The method of claim 20, further comprising sequencing the barcoded extension product or an intermediate thereof.

23. The method of claim 20, further comprising amplifying the barcoded extension product or the intermediate thereof prior to the sequencing.

24. The method of claim 1, further comprising determining: (i) the barcode sequence or a complement thereof, and (ii) all or a portion of a sequence of the barcoded nucleic acid fragment or a complement thereof.

25. The method of claim 1, wherein the tagmenting in (c) comprises contacting the barcoded nucleic acid molecule with a transposome comprising the transposon end sequence and a transposase.

26. The method of claim 25, wherein the transposase is a Tn5 transposase, a Mu transposase, a Tn7 transposase, a Vibrio species transposase, or a functional derivative thereof.

27. The method of claim 1, further comprising, prior to (a), partitioning a cell, cell bead, or nucleus comprising the mRNA analyte with the nucleic acid barcode molecule attached to the solid support.

28. The method of claim 27, wherein the barcode sequence comprises a partition-specific barcode sequence.
